(19) United States
(12) Patent Application Publication
(10) Pub. No.: US 2022/0133410 A1
(43) Pub. Date: May 5, 2022

(54) AN ARTIFICIAL INTELLIGENCE BASED SYSTEM FOR DETERMINING OPTIMAL BURR HOLE POINTS IN NEUROSURGERY

(71) Applicant: Innowayrg Arastirma Gelistirme Ve Danismanlik Hizmetleri Sanayi Ve Ticaret Anonim Sirketi, lstanbul (TR)

(72) Inventors: Serdar Baki ALBAYRAK, Istanbul (TR); Emrullah SAKU, Istanbul (TR); Onur YOLAY, Istanbul (TR)

(21) Appl. No.: 17/434,101

(22) PCT Filed: Jan. 8, 2020
(86) PCT No.: PCT/TR2020/050014

§ 371 Date: Aug. 26, 2021

(30) Foreign Application Priority Data
Sep. 15, 2019 (TR) 2019/13966

(51) Int. Cl. A61B 34/10; G06T 7/00; G16H 20/40; G16H 30/40

(52) U.S. Cl. CPC A61B 34/10; G06T 7/0012; G16H 20/40; G16H 30/40; G06T 2207/10081; G06T 2207/30016; G06T 2207/20084; A61B 2034/107; G06T 2207/10024; G06T 2207/10088; A61B 2034/105

(57) ABSTRACT

An artificial intelligence-based system for determining the appropriate burr-hole point (drilling holes in the skull) using cranial tomography and/or MR images and patient photographs in ventriculostomy, shunt and craniotomy procedures in neurosurgery operations.

### TECHNICAL FIELD

The invention relates to an artificial intelligence-based system for determining the appropriate burr-hole point (drilling holes in the skull) using cranial tomography and/or MR images and patient photographs in ventriculostomy, shunt and craniotomy procedures in neurosurgery operations.

### BACKGROUND OF THE INVENTION

In the neurosurgery practice, a suitable bone window is removed (craniotomy) or bore-hole (burr-hole drilling, trepanization) from the skull for surgical treatment of pathogens (tumors, abscesses, vascular tangles, hydrocephalus, etc.) in the skull and brain tissue.

The price of the currently used neuro-navigation systems is quite high. Furthermore, it takes time to install and use.

In the present technique, it is not possible to use neuro-navigation systems especially in emergency neurosurgery procedures (cerebral hemorrhage, hydrocephalus etc.).

There is no system that can be used both on mobile devices such as mobile phone tablets and more complicated computers.

In the literature, US patent application U.S. Pat. No. 6,267,769B1 relates to the subject matter “A surgical method and apparatus for accurately aligning the trajectory of, guiding of, and introducing or withdrawal of an instrument includes a base with a movable member. The base has a tubular shape. Positioned near the first end of the base is a seat. The seat is dimensioned to receive a movable member. A positioning member is used to move the movable member. The initial position of the movable member is determined using a scanning device, such as a CT scanner, frameless stereotaxy device or an MRI device. The movable member is elevated above the patient so that a burr hole does not have to be made in the patient to do the above described procedure. The second end has an opening therein and the tubular body is positioned between the seat and the second end. A flange near the second end is used to attach the base to the patient. The flange may also engage a plastic ring such that it can rotate or swivel with respect to the ring. The ring is attached to a flexible adhesive patch so which may be attached to the body. A portion or the entire positioning stem may be doped to make the positioning stem detectable by x-radiation and by the CT scanner. Arched bails can also be attached to the base for adjusting the trajectory alignment. An adapter externalizes burr holes and eliminates the need for burr hole. The externalizer forms a substitute burr hole away from the skull or body so tools which usually work only within a burr hole can be used without having to make a burr hole.”

The present application discloses the Trajectory guidance method and apparatus for use in magnetic resonance and computer tomographic scanners.

In the literature, US patent application US2016256069A1 relates to the subject matter “Circuits and computer program products onboard and/or adapted to communicate with an scanner that electronically recognize predefined physical characteristics of the at least one tool to automatically segment image data provided by the scanner whereby the at least one tool constitutes a point of interface with the system. The circuits and computer program products are configured to provide a User Interface that defines workflow progression for an image guided surgical procedure and allows a user to select steps in the workflow, and generate multi-dimensional visualizations using the predefined data of the at least one tool and data from images of the patient in substantially real time during the surgical procedure.”

Said application discloses MRI-guided diagnostic or interventional systems which may be particularly suitable for the placement/localization of interventional medical devices and/or therapies in the body.

In the literature, European patent application EP3466334A1 relates to the subject matter “Treatment trajectory guidance systems and methods are provided. In one embodiment, the method for treatment trajectory guidance in a patient's brain includes obtaining a three-dimensional (3D) brain model that includes a model of an anatomy, the model of the anatomy including a plurality of feature points; modifying the 3D brain model based on magnetic resonance (MR) data of the patient's brain from a magnetic resonance imaging (MRI) device to obtain a plurality of modified feature points on a modified model of the patient's anatomy in the patient's brain; displaying on a display a first planned trajectory for treating the patient's anatomy based on the plurality of modified feature points; and displaying, on the display, a first estimated treatment result for the first planned trajectory.”

In said embodiment, the method is disclosed using therapy trajectory guidance and, in particular, based on a high resolution magnetic resonance scan, using altered brain partitions and using the planned trajectory to guide treatment.

Due to the above-mentioned disadvantages, there has been a need to provide a new artificial intelligence-based system that allows the identification of appropriate hole points in the neurosurgery.

### DISCLOSURE OF THE INVENTION

From this position of the art, the object of the invention is to provide a new artificial intelligence-based system for determining the appropriate hole points to the skull in neurosurgery operations.

Another object of the invention is to provide a structure which enables a much faster use.

Another object of the invention is to provide a structure that minimizes installation and usage costs.

Another object of the invention is to provide a structure which provides the advantage of being used in emergency surgeries.

Another object of the invention is to provide a structure which can be used both in mobile devices such as mobile phone tablets and more complicated computers.

### DESCRIPTION OF THE FIGURES

FIG. 1 Schematic view of artificial intelligence based system for neurosurgery subject of the invention

### REFERENCE NUMBERS

1. Main Database
2. Analysis Module
   2.1 Artificial Intelligence Algorithm Module
   2.2 Algorithm Classification Module
3. Learned Database
4. Artificial Intelligence Software
5. Patient
6. Doctor
7. Patient's Head Photo
8. Tomography/MR Images

### DETAILED DESCRIPTION OF THE INVENTION

In this detailed description, the novelty of the invention is illustrated only by examples which will have no limiting effect on a better understanding of the subject.

The invention provides an artificial intelligence-based system for determining the appropriate burr-hole points in the skull in ventriculostomy, shunt and craniotomy procedures in neurosurgery operations, characterized in that, comprises the process steps of, obtaining tomography/MR images (8) of the patient, loading the obtained tomography/MR images (8) into the main database (1), taking a total of four digital photographs of the patient's (5) head on the right and left sides, front and back to obtain the patient's head photo (7), processing the patient's head photo (7) in the artificial intelligence software (4) and saving it in the main database (1), to obtain learned artificial intelligence model data by analyzing the patient's head photo (7) and tomography/MR images (8) in the analysis module (2) and determining the projection point of a point in the brain on the scalp, storing the obtained artificial intelligence model data in the learned database (3).

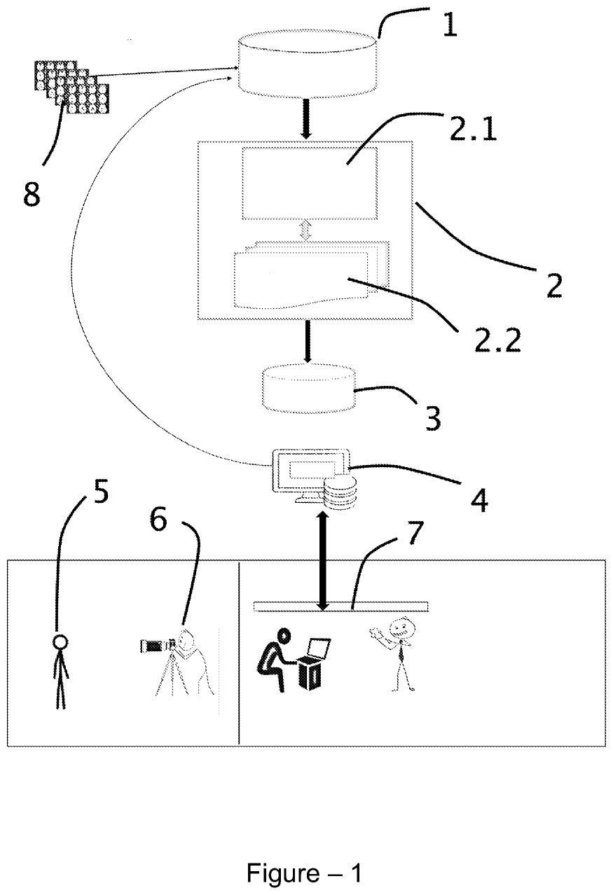

FIG. 1 shows a schematic view of artificial intelligence based system for neurosurgery subject of the invention.

The artificial intelligence software (4) of the present invention does not detect any pathology. Axial and/or sagittal, coronal digital tomography/MR images (8) of the at least 1000 patients (5) and the head of these patients (5) are located on the right and left sides a total of 4 digital photographs from the front and back of the patient's head (7) is obtained and the projection point of a point on the scalp is determined.

A total of at least 4 digital photographs or short-term video images of the head of said patients (5) on the right and left sides, front and back are uploaded to the artificial intelligence software (4). In an instant brain surgery attempt for the pathology of interest, the appropriate holes show the target point on the patient's skull relative to the reference point (the upper part of the auricle, the inion of the skull, the back protrusion (inion), etc.), which provides the surgeon with the advantage of a quick/immediate surgical procedure.

The artificial intelligence algorithm module (2.1) in said analysis module (2) extracts and analyzes tomography/MR images (8) from the main database (1). Patient (5) age, gender and so on. by adding the information to the patient (5) category and find the hole in the skull by finding the learned database (3) records. Thus, a learned data is produced.

In the said analysis module (2), the algorithm classification module (2.2) performs the function of selecting the most suitable algorithm for the patient (5) and classifying the algorithms obtained by studying.

In the operation of said system, firstly, a main database (1) which will be used by all artificial intelligence system is studied.

In the said main database (2), there is an architecture in which we can record the tomography/MR images (8) taken in the previous patients (5), the method followed in the patient (5) and the areas identified as Burr-holes.

The main database works in relational and no-sql architecture and is prepared for Big Data retention.

Said artificial intelligence software (4) first analyzes the tomography/MR images (8) by working with computer-vision algorithms and artificial intelligence algorithms within the artificial intelligence algorithm module (2.1).

In the development process of artificial intelligence, many algorithms are used to find the best result. (Example: Classification, RECURRENT NEURAL NETWORKS, RECURRENT NEURAL NETWORKS, CNN Algorithm, Bayes and Naive Bayes Algorithms, Decision Trees, Regression and Dimension Reduction Algorithms).

As a result of this analysis, the learned artificial intelligence model data is produced and recorded in the learned database (3).

Here, it is possible to ask questions to the learned data produced as a result. For example, it can instantaneously display Burr-hole points by asking questions to artificial intelligence software (4) over skull images.

1. An artificial intelligence-based system for determining appropriate burr-hole points in a skull in ventriculostomy, shunt and craniotomy procedures in neurosurgery operations, comprising the process steps of: obtaining tomography/MR images of a patient, loading the obtained tomography/MR images into a main database, taking a total of four digital photographs of the patient's head on the right and left sides, front and back to obtain the patient's head photo, processing the patient's head photo in artificial intelligence software and saving it in the main database, to obtain learned artificial intelligence model data by analyzing the patient's head photo and tomography/MR images in the analysis module and determining the projection point of a point in the brain on the scalp, and storing the obtained artificial intelligence model data in the learned database.

2. An artificial intelligence based system according to claim 1, comprising the processing step of the patient showing the target point relative to the reference point on the skull in order to determine the appropriate puncture points in the skull in the neurosurgical intervention for the relevant pathology.

3. An artificial intelligence based system according to claim 1, comprising the processing step of the tomography/MR images of an artificial intelligence algorithm module contained in said analysis module are extracted from the main database, and said patient is determined by age and gender and adding the information to the patient category and finding the hole opening points in the skull and saving it to the learned database.

4. An artificial intelligence based system according to claim 1, comprising the processing step of an algorithm classification module included in said analysis module includes the steps of classification of the algorithms obtained by studying and performing the function of selecting the most suitable algorithm for the patient.

5. An artificial intelligence based system according to claim 1, wherein said artificial intelligence algorithm module includes a computer vision algorithm and an artificial intelligence algorithm.

6. An artificial intelligence based system according to claim 1, wherein said artificial intelligence software is installed in the computer or mobile device.
